(19) United States
(12) Patent Application Publication
(10) Pub. No.: US 2025/0248588 A1
(43) Pub. Date: Aug. 7, 2025

(54) SYSTEMS AND METHODS FOR MEDICAL DEVICE INTUBATION

(71) Applicant: Noah Medical Corporation, San Carlos, CA (US)

(72) Inventors: Matthew Robert Penny, Holly Springs, NC (US); Jason Joseph Hsu, Mountain View, CA (US); Enrique Romo, Danville, CA (US); Leobardo Centeno, Ceres, CA (US); Hugo Quintero, San Pablo, CA (US); Hasan Tutkun Sen, Redwood City, CA (US); Andres Martinez Guerra, Redwood City, CA (US); Andrew Robert Barkan, San Carlos, CA (US); Julia Toye, San Francisco, CA (US); Maziyar Keshtgar, San Bruno, CA (US); Alan Fridman Landeros, Fremont, CA (US)

(21) Appl. No.: 19/058,666

(22) Filed: Feb. 20, 2025

Related U.S. Application Data
(63) Continuation of application No. PCT/US2023/073940, filed on Sep. 12, 2023.
(60) Provisional application No. 63/375,436, filed on Sep. 13, 2022.

(51) Int. Cl. A61B 1/00; A61B 1/005

(52) U.S. Cl. CPC A61B 1/00135; A61B 1/00006; A61B 1/00133; A61B 1/00154; A61B 1/0052; A61B 1/0055

(57) ABSTRACT

An overtube device is provided. The overtube device comprises features to form a first lumen for passing through a first scope during intubation. The first lumen is deformable for creating a second lumen for passing through a second scope. The second scope has a diameter greater than a diameter of the first scope.

### CROSS-REFERENCE

This application is a continuation of International Application No. PCT/US2023/073940, filed on Sep. 12, 2023, which claims priority to U.S. Provisional Patent Application No. 63/375,436, filed on Sep. 13, 2022, which is entirely incorporated herein by reference.

### BACKGROUND OF THE INVENTION

Endoscopy procedures use an endoscope to examine the interior of a hollow organ or cavity of the body. Unlike many other medical imaging techniques, endoscopes are inserted into the organ directly. Flexible endoscope that can deliver instinctive steering and control is useful in diagnosing and treating diseases that are accessible through any natural orifice in the body. Depending on the clinical indication, the endoscope may be designated as bronchoscope, ureteroscope, colonoscope, gastroscope, ENT scope, and various others. For example, a flexible colonoscope may be intubated to transverse colon for diagnosis and/or surgical treatment.

Certain endoscopes such as colonoscopes or gastroscopes may have relatively greater size and/or stiffness (compared to other types of endoscopes), making navigating tortuous anatomy can be more challenging. For instance, clinicians may use an overtube to ease intubation thereby providing a low friction surface and a defined trajectory to guide the intubation of the colonoscope. In some cases, reduction method may be adopted to ease the intubation by reducing the length and tortuosity of the colon by anchoring to the colon at the distal end and applying tension. However, it can be challenging to use overtubes for intubation. One such challenge in the current colon intubation process is the large size of the overtube device (i.e., balloon overtube) used for making passage for the inner endoscope and assisting in intubation.

### SUMMARY

A need exists to improve the intubation process of an endoluminal endoscopic device. The present disclosure addresses the above needs by providing an improved overtube for endoluminal device intubation. In particular, the overtube device of the present disclosure may ease the intubation process without reduced size or dimension (compared to conventional intubation process or devices).

In an aspect, a device for intubating an endoscope into a subject is provided. The device comprises: a flexible overtube comprising features to form a first lumen for passing through a first scope during intubation. The first lumen is deformable for creating a second lumen for passing through a second scope, and the second scope has a diameter greater than a diameter of the first scope.

In some embodiments, a diameter of the first lumen is smaller than a diameter of the second lumen. In some embodiments, the features comprise an expandible tubular structure to adjust a dimension of the first lumen or the second lumen. In some cases, the expandible tubular structure is a layflat tube construction with a pleat along an axial direction. In some cases, the expandible tubular structure is a foldable layflat tube construction. In some instances, the foldable layflat tube construction has separable edges that are engaged with aid of one or more active engagement features. In some instances, the foldable layflat tube construction adjusts a diameter of the first lumen to create the second lumen with aid of one or more passive engagement features.

In some embodiments, the first lumen and the second lumen are two channels separated by a lumen separator of the flexible overtube. In some embodiments, the second scope is a robotic scope. In some cases, the robotic scope comprises a handle portion releasably coupled to a robotic support. In some cases, the handle portion of the robotic scope is coupled to the robotic support after the robotic scope is inserted through the second lumen assuming a tortuous shape. In some cases, the robotic scope is initialized by removing slack in one or more pull wires for controlling articulation of a bending section of the robotic scope while the bending section conforms to an internal environment within the subject.

In another aspect, a method for intubating a robotic endoscope into a subject is provided. The method comprises: (a) performing an initial intubation to reach a target site within a body of the subject with a first scope and an overtube device, where the first scope is engaged with a first lumen of the overtube device; (b) withdrawing the first scope and inserting a second scope into a second lumen of the overtube device to reach the target site, where the second scope is a robotic scope having a diameter greater than a diameter of the first scope; (c) coupling a handle portion of the second scope to an instrument driving mechanism (IDM) and performing initialization of the second scope while the second scope is within the body of the subject.

In some embodiments, the initialization comprises removing a slack in one or more pull wires of the second scope. In some cases, the one or more pull wires are driven by the IDM to control an articulation of a bending section of the second scope in one or more degrees of freedom.

In some embodiments, the method further comprises monitoring a tension in one or more pull wires corresponding to one degree of freedom. In some cases, the method further comprises comparing a difference of the tension in the one or more pull wires against a predetermined threshold. In some examples, the method further comprises controlling one or more actuators of the IDM based on the tension or the difference of the tension.

In some embodiments, the first lumen is deformable for creating a second lumen. In some embodiments, the overtube device comprises an expandible tubular structure to adjust a dimension of the first lumen or the second lumen. In some cases, the expandible tubular structure is a layflat tube construction with a pleat along an axial direction. In some cases, the expandible tubular structure is a foldable layflat tube construction. In some cases. the first lumen and the second lumen are two channels separated by a lumen separator of the overtube device.

In a further aspect, a method is provided for initializing a robotic endoscope inside a subject. The method comprises: (a) while the robotic endoscope is placed inside the subject, driving a pair of pull wires at a constant velocity by an instrument driving mechanism, wherein the pair of pull wires are actuated to control an articulation of a bending section of the robotic endoscope corresponding to a first degree of freedom; (b) comparing a difference of tension in the pair of pull wires to a first threshold, and changing a movement of the pair of pull wires to decrease the difference of tension when the first threshold is reached; and (c) comparing a tension in the pair of pull wires to a second threshold and when the tension in either one of the pair of pull wires reaches the second threshold, stopping the movement of the corresponding pull wire.

In some embodiments, the second threshold is higher than the first threshold. In some embodiments, (a)-(c) are repeated for a pair of pull wires corresponding to a second degree of freedom. In some cases, (a)-(c) are performed concurrently for the first and the second degree of freedom. Alternatively (a)-(c) are performed sequentially for the first and the second degree of freedom.

In some embodiments, the robotic scope comprises a handle portion releasablely coupled to the instrument driving mechanism. In some cases, the instrument driving mechanism is supported by an end effector of a robotic arm. In some embodiments, the robotic scope comprises a flexible elongated member and a current shape, position or orientation of the elongated member is unknown.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 schematically shows an example of an intubation process.

FIG. 2 shows an example of an expandable overtube that is shaped to wrap around an endoscope.

FIGS. 3A-3C show an example of an overtube with a temporary longitudinal seam, providing for an expandable overtube device.

FIG. 4 shows an example of an overtube providing for volume reduction via internal vacuum during initial intubation.

FIG. 5 shows an example of an overtube device including a shrinkable overtube.

FIGS. 6A-B shows an example of an overtube device with a flexible multilumen construction.

FIG. 7 illustrates an example of a flexible endoscope, in accordance with some embodiments of the present disclosure.

FIG. 8 shows a robotic endoscope including a handle portion and a flexible elongate member.

FIG. 9 shows an example of an instrument driving mechanism providing mechanical interface to the handle portion of the robotic endoscope.

FIG. 10 shows an example of a distal tip of an endoscope.

FIG. 11 shows an example distal portion of the catheter with integrated imaging device and the illumination device.

FIG. 12 shows an example of an algorithm for initialization of the robotic endoscope (e.g., robotic colonoscope) to an instrument drive mechanism (IDM).

FIG. 13 and FIG. 14 show an example of an instrument driving mechanism (IDM) providing a mechanical interface to a handle portion of a robotic endoscope.

FIG. 15 shows an example of a robotic colonoscope.

FIG. 16 shows an example of a tip for a robotic colposcope device.

### DETAILED DESCRIPTION OF THE INVENTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The embodiments disclosed herein can be combined in one or more of many ways to provide improved diagnosis and therapy to a patient. The disclosed embodiments can be combined with existing methods and apparatus to provide improved treatment, such as combination with known methods of pulmonary diagnosis, surgery and surgery of other tissues and organs, for example. It is to be understood that any one or more of the structures and steps as described herein can be combined with any one or more additional structures and steps of the methods and apparatus as described herein, the drawings and supporting text provide descriptions in accordance with embodiments.

While exemplary embodiments will be primarily directed at a device or system for colonoscope or gastroscope, one of skill in the art will appreciate that this is not intended to be limiting, and the devices described herein may be used for other therapeutic or diagnostic procedures and in various anatomical regions of a patient's body. The provided device or system can be utilized in urology, gynecology, rhinology, otology, laryngoscopy, gastroenterology with the endoscopes, combined devices including endoscope and instruments, endoscopes with localization functions, one of skill in the art will appreciate that this is not intended to be limiting, and the devices described herein may be used for other therapeutic or diagnostic procedures and in other anatomical regions of a patient's body, such as such as brain, heart, lungs, intestines, eyes, skin, kidney, liver, pancreas, stomach, uterus, ovaries, testicles, bladder, ear, nose, mouth, soft tissues such as bone marrow, adipose tissue, muscle, glandular and mucosal tissue, spinal and nerve tissue, cartilage, hard biological tissues such as teeth, bone and the like, as well as body lumens and passages such as the sinuses, ureter, colon, esophagus, lung passages, blood vessels and throat, and various others, in the forms of: NeuroendoScope, EncephaloScope, OphthalmoScope, OtoScope, RhinoScope, LaryngoScope, GastroScope, EsophagoScope, BronchoScope, ThoracoScope, PleuroScope, AngioScope, MediastinoScope, NephroScope, GastroScope, DuodenoScope, CholeodoScope, CholangioScope, LaparoScope, AmioScope, UreteroScope, HysteroScope, CystoScope, ProctoScope, ColonoScope, ArthroScope, SialendoScope, Orthopedic Endoscopes, and others, in combination with various tools or instruments.

The systems and apparatuses herein can be combined in one or more of many ways to provide improved diagnosis and therapy to a patient. Systems and apparatuses provided herein can be combined with existing methods and apparatus to provide improved treatment, such as combination with known methods of pulmonary diagnosis, surgery and surgery of other tissues and organs, for example. It is to be understood that any one or more of the structures and steps as described herein can be combined with any one or more additional structures and steps of the methods and apparatus as described herein, the drawings and supporting text provide descriptions in accordance with embodiments.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used herein, the terms distal and proximal may generally refer to locations referenced from the apparatus, and can be opposite of anatomical references. For example, a distal location of a primary shaft or catheter may correspond to a proximal location of an elongate member of the patient, and a proximal location of the primary sheath or catheter may correspond to a distal location of the elongate member of the patient.

### Overtube Intubation for Robotic Endoscope System

For diagnostic and therapeutic procedures in bodily lumen, clinicians have traditionally intubated with manual endoscopes. In the colon, manual colonoscopy is able to successfully reach the target such as the cecum (the end of the large intestine) relatively easily (e.g., in less than 10 minutes) for most patients. However, the intubation process can be more challenging for robotic colonoscope system due to the increase size/dimension of the robotic colonoscope device. During an assisted or autonomous intubation process with robotic colonoscopes, overtubes may be utilized to ease the intubation. An overtube is a sleeve-like equipment usually made of semi-rigid plastic or silicone rubber, designed to assist endoscopy. An overtube of a larger diameter than that of an endoscope is needed for providing a route through the gastrointestinal (GI) tract.

Current intubation workflow for robotic colonoscopes may suffer from increased cost, intubation time and the potential risk of perforation while under control without haptic feedback to the user. A typical workflow for robotic colonoscope may comprise docketing the colonoscope to the robotic system prior to intubation. As an example, current intubation workflow for robotic colonoscope may comprise engaging the overtube with the colonoscope, coupling the engaged colonoscope and overtube to a robotic driving mechanism, then intubating the engaged colonoscope and overtube together (controlled by a robotic system) into the subject lumen via repeated process of inflating balloon, reducting the colon (e.g., shorten and straighten colon), anchoring colonoscope with the tip, deflating balloon, advancing the overtube to the tip of the colonoscope, and the process is repeated until the device reaches the target site. However, the above workflow can be time consuming, and may suffer potential risk of perforation without haptic feedback to the user.

The present disclosure addresses the above drawbacks by providing an improved workflow for intubating a robotic colonoscope. In particular, the workflow may comprise coupling a robotic colonoscope to a robotic drive mechanism (instrument driving mechanism (IDM)) after the colonoscope has been intubated and with the colonoscope in a position and orientation dictated by the curvature of the patient's anatomy. Coupling a colonoscope to a robotic driving mechanism is challenging because once the colonoscope is inside the patient, particularly in a position and orientation dictated by the curvature of the patient's anatomy, it is difficult to know the initial colonoscope configuration and position. In order to control an endoscope (e.g., colonoscope), initialization for coupling the endoscope to the robotic driving mechanism is required as a robotic system needs to know the initial colonoscope configuration and minimize the backlash between the driving mechanism and the tip. Failing on the initialization may result in unintended motion and trauma to the patient.

However, traditional initialization method may not be suitable for an intubated endoscope. There are generally two initialization approaches for robotic instruments: one approach is to place the device in a fixture that maintains the tool in a known position during the backlash removal. A second approach is to move toward the limits of the different degrees of motion and then move away from those limits a predetermined amount for initialization/calibration. The above approaches are not suitable for docketing an endoscope device that is already intubated or placed inside a patient as they are either impractical (requiring an extra fixture to place the endoscope in a tensioning fixture) or may potentially injure the patient (instrument/colonoscope has to move to its limits).

Methods and systems herein may provide an improved docketing method for an intubated colonoscope thereby preventing trauma to a patient when coupling the robotic colonoscope to the robotic drive mechanism for a robotic endoluminal surgical procedure.

In an aspect of the present disclosure, a method of intubating robotic endoscope device with an overtube is provided. The method may provide an improved workflow comprising coupling a robotic colonoscope to a robotic drive mechanism (instrument driving mechanism (IDM)) after the colonoscope has been intubated and with the colonoscope in a position and orientation dictated by the curvature of the patient's anatomy. The method may comprise intubating a robotic endoscope with an overtube, then coupling the intubated robotic endoscope to an IDM once it is inside a subject's body. The overtube may have a reduced dimension details of which are described later herein.

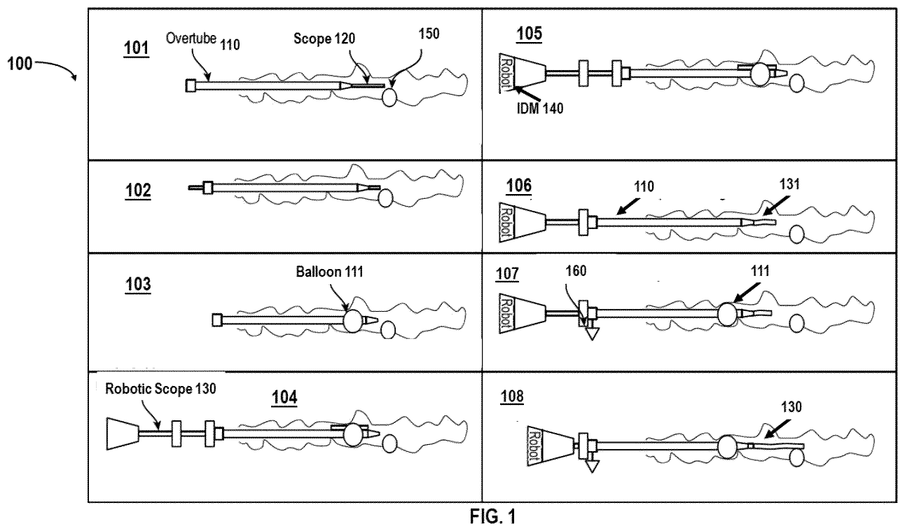

FIG. 1 schematically shows an example of an intubation process 100 for an endoscope device. The endoscope device may be a robotic endoscope device such as a robotic colonoscope. The robotic endoscope device may have an increased dimension due to additional components for the robotic control or robotic features. For instance, the robotic endoscope may have a larger-than-normal diameter.

The intubation workflow 100 may comprise intubating 101 with a first scope 120 (e.g., manual scope, manual colonoscope), placing an overtube 110, and advancing the first scope 120 and the overtube 110 together until reaches a position. The first scope 120 can be any available endoscope that can be manually inserted into a patient lumen. The first scope 120 may have a dimension smaller than a dimension of a second scope such as a robotic scope that is to be intubated. For example, a diameter of the scope 120 may be smaller than a diameter of a robotic scope 130. The intubation step 101 can be a convention process such as the bending section of the scope 120 is advanced over the tip of the overtube 110 and then the overtube is advanced to the tip of the scope 120 until they reach a target site 150. The overtube 110 may have a reduced dimension and will be described in detail later herein.

Once the scope tip reaches the target site or site of interest 150, the workflow may comprise an operation 102 of advancing the overtube 110 along the bending section of the scope 120 to place a balloon 111 proximal to the tip of the scope 120. Next, the workflow may comprise an operation 103 of inflating the balloon 111 and removing the scope 120 while leaving the overtube in place. In some cases, the colon may be reducted.

Next, a second scope such as a robotic endoscope (e.g., gastroscope or robotic colonoscope) 130 is inserted through the overtube 104. In some cases, the robotic endoscope 130 may be inserted manually. Once the tip of the robotic endoscope 130 is placed at the site of interest, the proximal end of the robotic endoscope 130 may be connected to a robotic drive mechanism or instrument driving mechanism (IDM) 140. The workflow may proceed with an operation 106 of deflating the balloon of the overtube and the overtube may be pulled in the proximal direction to expose the bending section 131 of the robotic endoscope. In the next operation 107, the balloon 111 of the overtube is inflated and the overtube may be grounded such as via a grounding mechanism 160 located at a proximal end of the overtube.

The robotic endoscope 130 may then be capable of performing any controlled operations 108 at the site of interest. In some cases, the robotic operations may comprise an improved initialization process provided by the present disclosure. Once the initialization process is completed, a user or operator may control the robotic colonoscope 130 during a procedure. The colonoscope may be operated by a user robotically such as from a surgeon console which is positioned away from the patient. Once the procedure is completed, the robotic colonoscope is disconnected from the robotic drive mechanism and is removed along with the overtube from the patient. The overtube may be single-use or disposable. The robotic colonoscope 130 may be single-user or disposable. Alternatively, at least part of the robotic colonoscope is reusable.

Initialization for an intubated endoscope (i.e., endoscope is already inserted into a patient's anatomy) can be challenging. As described above, conventional methods are not suitable for docketing an endoscope device that is already intubated or placed inside a patient (where the scope assumes a tortuous shape of the pathway) as they are either impractical (requiring an extra fixture to place the endoscope in a tensioning fixture) or may potentially injure the patient (instrument/colonoscope has to move to its limits).

Methods and systems herein may provide an improved docketing method for an intubated colonoscope thereby preventing trauma to a patient when coupling the robotic colonoscope to the robotic drive mechanism for a robotic endoluminal surgical procedure.

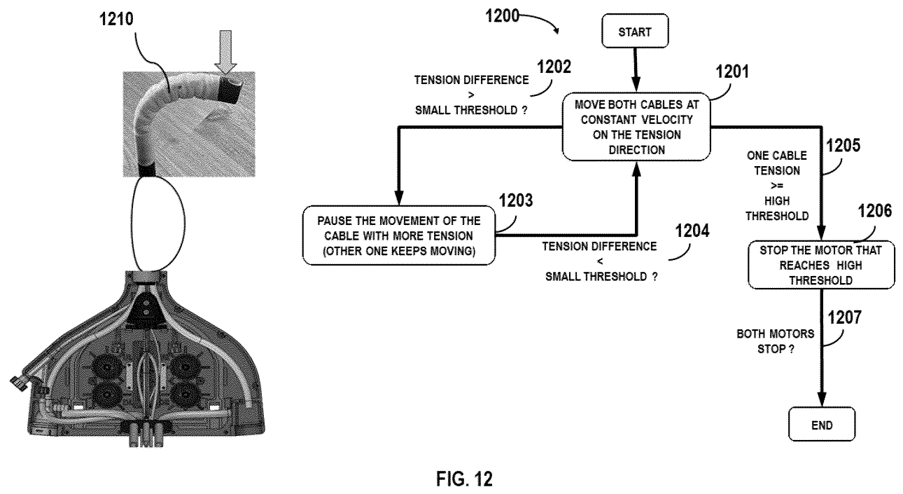

FIG. 12 shows an example of the algorithm 120 for initialization of the robotic endoscope (e.g., robotic colonoscope) to an instrument drive mechanism (IDM). In some embodiments, the robotic system may utilize motor currents from the IDM to sense a transmission load and pull wires (cables) in the robotic endoscope (e.g., colonoscope, gastroscope) to transmit the motion.

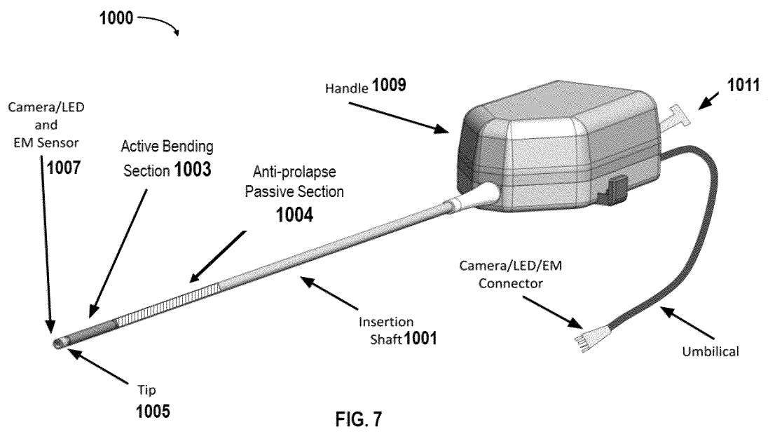

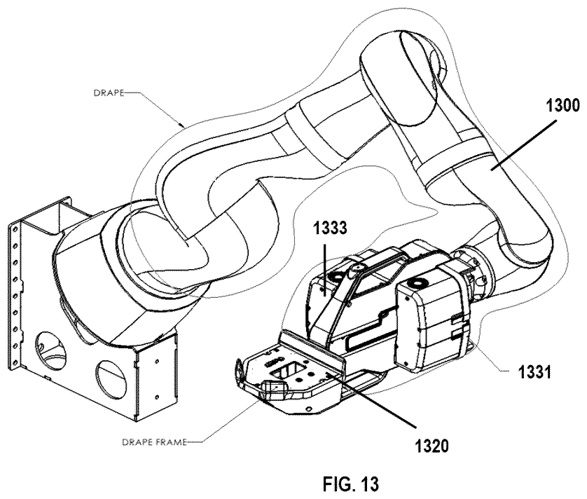

The method may be applied to any suitable robotic endoscope system. The robotic endoscope system can be the same as those described in FIGS. 13-15 and/or FIGS. 7-11. As shown in FIGS. 13-15, a robotic endoscope (e.g., colonoscope, gastroscope) 1500 can be releasably coupled to an instrument driving mechanism 1320. The instrument driving mechanism 1320 may be mounted to the arm of the robotic support system 1300 or to any actuated support system as described elsewhere herein. The instrument driving mechanism may provide mechanical and electrical interface to the robotic endoscope 1500. The mechanical interface may allow the robotic endoscope 1110 to be releasably coupled to the instrument driving mechanism. For instance, the handle portion 1501 of the robotic gastroscope can be attached to the instrument driving mechanism via quick install/release means, such as magnets and spring-loaded levels. In some cases, the robotic gastroscope may be coupled or released from the instrument driving mechanism manually without using a tool.

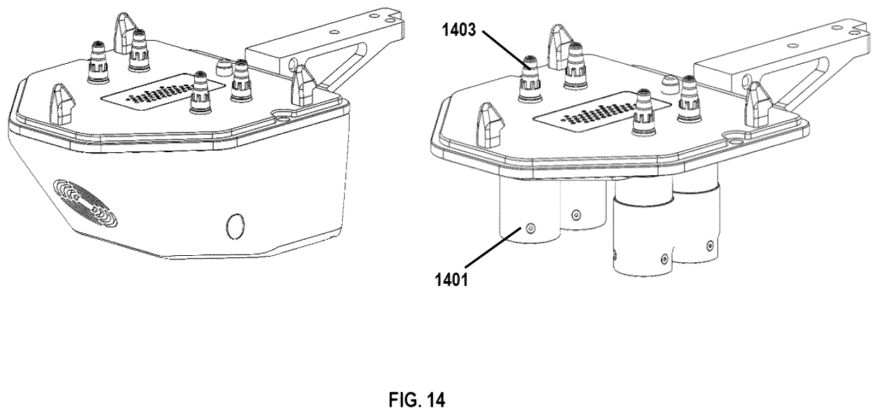

FIG. 13 and FIG. 14 show an example of an instrument driving mechanism (IDM) 1320 providing a mechanical interface to the handle portion of the robotic endoscope 1500. In some cases, the system may comprise an IDM 1320 for a robotic endoscope and one or more IDMs 1331, 1333 for one or more instruments (e.g., suturing instrument) that are attached to the robotic arm 1300. As shown in the example, the instrument driving mechanism (IDM) 1320 for the robotic endoscope 1500 may comprise a set of motors 1401 that are actuated to rotationally drive a set of pull wires of the flexible robotic endoscope or catheter. The handle portion of the catheter assembly may be mounted onto the instrument drive mechanism 1320 so that its pulley assemblies or capstans of the IDM interface 1511 are driven by the set of motors 1401. The number of pulleys may vary based on the pull wire configurations. In some cases, one, two, three, four, or more pull wires may be utilized for articulating the bending section 1505 of the flexible robotic endoscope or catheter. Similarly, the instrument driving mechanism (IDM) 1331 for an instrument (e.g., suturing instrument) may comprise a set of motors that are actuated to rotationally drive a set of pull wires of the instrument.

The handle portion may be designed allowing the robotic gastroscope to be disposable at reduced cost. For instance, classic manual and robotic gastroscopes may have a cable in the proximal end of the gastroscope handle. The cable often includes illumination fibers, camera video cable, and other optional sensor fibers or cables such as electromagnetic (EM) sensors, or shape sensing fibers. Such complex cable can be expensive, adding to the cost of the gastroscope. The provided robotic gastroscope may have an optimized design such that simplified structures and components can be employed while preserving the mechanical and electrical functionalities. In some cases, the handle portion of the robotic gastroscope may employ a cable-free design while providing a mechanical/electrical interface to the catheter. The robotic scope may comprise a tip 1507 with integrated components. Details about the robotic endoscope and the distal tip are described later herein.

Referring back to FIG. 12, a robotic manipulator may comprise an instrument drive mechanism (IDM) housing a plurality of motors (e.g., four motors) to facilitate independent control over each steering element in the accompanying robotic endoscope (e.g., colonoscope). The robotic colonoscope can be the same as those described elsewhere herein. For instance, the robotic colonoscope may have a long, flexible shaft with a steerable distal tip and a proximal handle for coupling to the instrument drive mechanism (IDM). In some embodiments of the initialization process, the colonoscope may be initialized to the instrument drive mechanism by using motor currents from the IDM to sense the transmission load and pull wires (e.g., cables) in the colonoscope to transmit the motion.

The algorithm 1200 may allow slack removal on the antagonistic transmission of a flexible-robot bending-section 1210 while allowing the bending section 1210 to conform to the environment where it is originally placed (e.g., the bending section having a position, orientation and/or shape conforming to the curvature or the tortuous shape of the patient's anatomy).

The algorithm assumes that a degree of freedom (DOF) is driven antagonistically by two actuators. For instance, two pull wires may be driven by two actuators respectively, corresponding to one DOF (e.g., opposite directions). The two actuators may be independent and are controlled to tension and slack the two pull wires in concert with one another through a control algorithm. In some cases, both pull wires are tensioned to set an initial tension state. In some cases, one pull wire is tensioned while the other pull wire is slacked to provide movement. In some cases, both pull wires may be slacked to facilitate removal of the endoscope device from a tortuous pathway by allowing the bending section to conform to the anatomy passively. In alternative embodiments, one DOF may be driven by one actuator. For instance, two pull wires may be coupled to a driven pulley antagonistically to drive the bending section motion (i.e., in one degree of freedom) such that rotation of the pulley provides tension to one pull wire when it rotates in one direction while slacking the second pull wire.

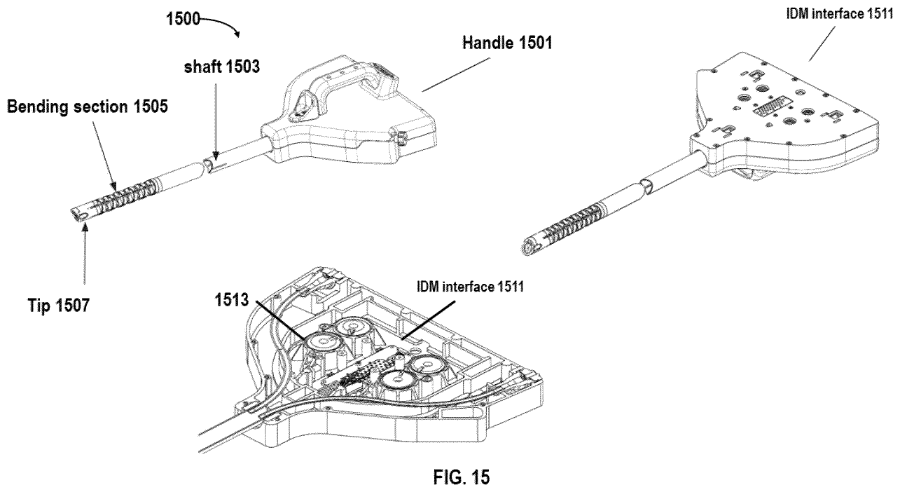

As shown in FIG. 14 and FIG. 15, the colonoscope 1500 comprises a handle 1501 for attaching to the IDM via an IDM interface 1511, a flexible elongate shaft 1503 and a steerable bending section 1505. In the illustrated example, the colonoscope has four cable transmissions (i.e., pull wires) that terminate distal to the steerable bending section and route through the elongate shaft to the proximal capstans 1513 housed in the handle. When the handle is attached to the IDM (shown in FIG. 14), the capstans 1513 may align with the output shafts 1403 of the IDM motors 1401 such that rotation of the output shaft creates a corresponding rotation in the colonoscope capstan. Rotation of the colonoscope capstan in one direction relieves cable tension, while rotation in the other direction increases cable tension. In some cases, the cables in the colonoscope distal portion are arranged spatially so that a pair of cables (corresponding to one degree of freedom of the bending section such as yaw) exist on a plane that intersects the neutral axis of the bending section. The other two cables may exist on an orthogonal plane that intersects the neutral axis of the bending section (e.g., pitch). A pair of cables corresponding to one degree of freedom may be driven by a pair of actuators (motors).

The initialization of the colonoscope to the IDM may comprise removing any slack existed in the pull wire (cables). As shown in FIG. 12, the initialization algorithm 1200 may comprise commanding a pair of actuators to move in the tension direction respectively 1201. In some cases, the pair of actuators may move in their actuation direction at a constant velocity while the exerted load is monitored. The constant velocity may be within a predetermined range to ensure the initialization process is performed quickly while not breaking the cable. The predetermined range may be based on empirical data. In some cases, the constant velocity may vary based on different bending section configuration (e.g., connection/layout of the pull wires), may or may not be the same across different degrees of freedom (e.g., the constant velocity associated with pitch may or may not be the same as the constant velocity associated with yaw) or may be adjusted based on different use applications. In some cases, the constant velocity may be configurable by a user via a user interface. Alternatively, the constant velocity may be adjusted or determined automatically (e.g., during calibration process prior to inserting the endoscope into a subject's body).

The system may monitor the exerted load based on any suitable sensor data or measurements. In some embodiments, the system may monitor the exerted load based on motor current of each motor. This beneficially allows for load measurement without requiring extra components.

The monitoring may comprise continuously determining if the difference in load (i.e., tension difference) in the pair of cables greater than a predetermined “difference threshold” (e.g., small threshold 1202). If the tension difference is greater than the predetermined difference threshold, the actuator with greater load is paused while the other actuator continues the movement 1203. By constantly comparing the tension difference with the different threshold or small threshold 1202 and adjusting the tension difference accordingly, the tension difference of the pair of cables may maintain within an acceptable range thus the motion of the bending section/distal portion of the endoscope is maintained within an acceptable range from its current position/orientation. The small threshold 1202 may be predetermined based on empirical data. In some cases, the small threshold 1202 may vary based on different bending section configuration (e.g., connection/layout of the pull wires), may or may not be the same across different degrees of freedom (e.g., the small threshold associated with pitch may or may not be the same as the small threshold associated with yaw) or may be adjusted based on different use applications. In some cases, the small threshold may be configurable by a user via a user interface. Alternatively, the small threshold may be adjusted automatically (e.g., during calibration process prior to inserting the endoscope into a subject's body).

As the other actuator keeps moving, the difference in load or tension difference decreases and when the tension differences is detected to be at or below the “difference threshold” 1204, both the actuators may return to move in their actuation directions again 1201. This process may be repeated to increase the load or tension in the pair of cables until the tension in at least one of cables reaches (e.g., detected to be at or greater than) a predetermined high threshold 1205. The high threshold may refer to a load threshold higher than the small threshold. The high threshold may be determined based on empirical data. In some cases, the high threshold 1205 may vary based on different bending section configuration (e.g., connection/layout of the pull wires), may or may not be the same across different degrees of freedom (e.g., the high threshold associated with pitch may or may not be the same as the high threshold associated with yaw) or may be adjusted based on different use applications (e.g., target site environment, etc.). In some cases, the high threshold may be configurable by a user via a user interface. Alternatively, the high threshold may be adjusted automatically (e.g., during calibration process prior to inserting the endoscope into a subject's body).

If the tension in at least one of cables reaches (e.g., detected to be at or greater than) a predetermined high threshold, the corresponding actuator may be stopped from moving while the other actuator keeps movement until the tension also reaches the high threshold 1207. The algorithm may instruct the other actuator to stop movement and the initialization process is completed.

In some cases, once the slack is removed for a pair of cables (e.g., corresponding to a first degree of freedom), the above process may be repeated for the other pair of cables (e.g., corresponding to a second degree of freedom). In some cases, the process may be conducted on two or more DOFs concurrently to reduce the overall initialization time. Alternatively, the process may be conducted on different DOFs sequentially to avoid cross-talk between the DOFs during the initialization.

The above algorithm may beneficially remove the slack existed in the pull wires or cables while the bending section or the robotic colonoscope is placed inside of a subject's body. This above method may not require knowledge of an initial (current) shape, orientation or position of the robotic colonoscope. Although the above algorithm is described in the context of robotic scope or colonoscope intubation process, it should be noted that the above algorithm can be executed or applied in any scenarios when initialization of a robotic scope is desired while the scope is placed inside of a subject's body regardless the types of scope. For example, during surgical operation, if the system's operation is paused due to safety reason or any type of failure, the robotic scope may be initialized from it is current position/orientation without damaging the tissue by performing the method as described above.

### Overtube Device with Reduced Dimension

As described in the intubation process of FIG. 1, it can be difficult to intubate or insert the second scope (e.g., robotic endoscope, gastroscope) to the transverse colon as the second scope may have sizes or stiffness different from the first endoscope (e.g., standard colonoscope or manual scope). For example, the gastroscope may have a diameter larger than a standard colonoscope (e.g., 18 mm vs. 13 mm), and may be stiffer and shorter (e.g., 80 cm vs 160 cm) compared to the standard colonoscope. As the diameter difference between the second scope (e.g., robotic endoscope, gastroscope) and the first scope (e.g., standard colonoscope or manual scope) can be large (e.g., about 5 mm or greater), the overtube for delivering the second scope has to be large enough to accommodate the second scope. For instance, the diameter of the overtube is greater than (>) that of the second scope's outer diameter. However, attempting to intubate the colon with such overtube along with the first scope having a smaller diameter can introduce potential risks and challenges. For example, the gap between the overtube and the standard colonoscope may cause the overtube to catch on tissue and prevent its advancement. Additionally, one challenge users experience while intubating with a balloon assisted overtube is the large size of the overtube device.

The present disclosure provides a novel overtube with reduced size during initial intubation that can be used with an endoscope or colonoscope to ease insertion, manipulation, and retraction of an endoscope during colonoscopy upper gastrointestinal (GI) tract endoscopy, gastric endoscopy, small bowel endoscopy or other procedures. In some embodiments, the overtube herein may be capable of changing a dimension of the inner space for passing scopes of different diameters. In particular, the present disclosure provides an overtube device with a reduced size for overtube intubation with the capability to adapt to scopes with different diameters. In some cases, an outer diameter of the overtube during an initial intubation process may be smaller than the outer diameter of the overtube during intubation for the larger scope. The term “initial intubation” as utilized herein may refer to the intubation process with the smaller diameter scope (e.g., operation 101 in FIG. 1). For instance, the overtube device may allow expansion to deliver a scope (e.g., a gastroscope or robotic scope) with a size greater than that of the scope for the initial intubation. In some cases, a dimension of an inside space of the overtube for passing the scope during an initial intubation process may be smaller than the dimension of the inside space of the overtube during the intubation for the larger scope.

In an aspect of the present disclosure, an expandable overtube device with reduced size is provided. The overtube device may have a substantially tubular shape and may have a compact configuration. The overtube device may have a deformable elongate body to adapt to scopes with different diameters. In some embodiments, the overtube device may be delivered inside of the subject's anatomy (e.g., colon) and have a first diameter (e.g., outer diameter or inner diameter) during the intubation process, and may be expanded radially to allow the passage of a device that has a larger diameter than the first diameter of a scope that is used for intubation.

In some cases, the overtube may be pleated along the diameter such that the relaxed state of the tube may include one or more folds and create a temporary lumen with a small diameter. The lumen may refer to a space inside a substantially tubular structure. The term “lumen” as utilized herein may refer to a space inside a substantially tubular structure, a partial lumen i.e., a space inside a partial tubular structure (e.g., wall is not enclosed), or a space defined by a substantially tubular structure with any suitable cross-sectional shape or dimensions. The temporary lumen may allow for passing through a first scope with a smaller diameter during intubation. In an active state, the inner diameter of the overtube may be expanded, eliminating the pleat(s) to pass the second scope (e.g., gastroscope). The active state may allow for the insertion of an object (e.g., gastroscope) that is larger than the relaxed state of the sheath. The pleated sheath may expand around the larger object, allowing it to pass and conforming to fit the diameter of objects.

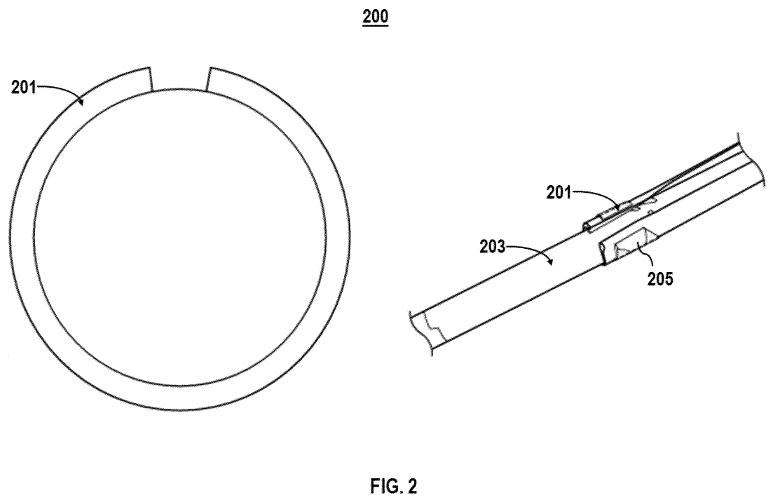

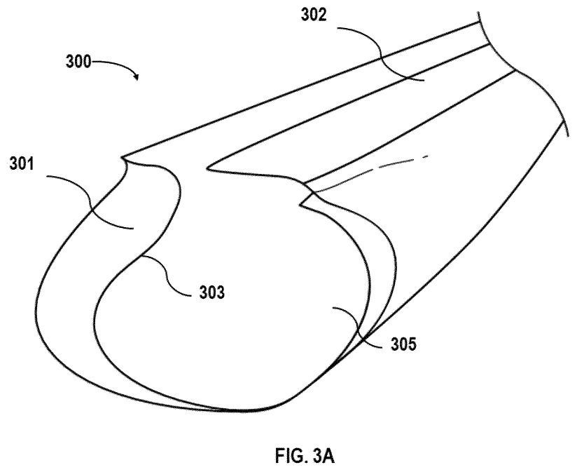

FIG. 2 shows an example 200 of an expandable overtube device used for intubation. The overtube device 201 may be a pleated, radially expanding overtube. In some cases, the overtube device 201 may have a layflat tube construction where the inner diameter of the layflat is large enough to pass a second scope (e.g., gastroscope) 203. The expandable overtube 201 may be shaped to wrap around an endoscope 203 without fully encapsulating where the pleat feature may allow the overtube to adapt to scopes with different diameters. as shown in the example, the pleat feature may be along an axis direction of the layflat tube construction to allow the layflat tube construction to expand radially.

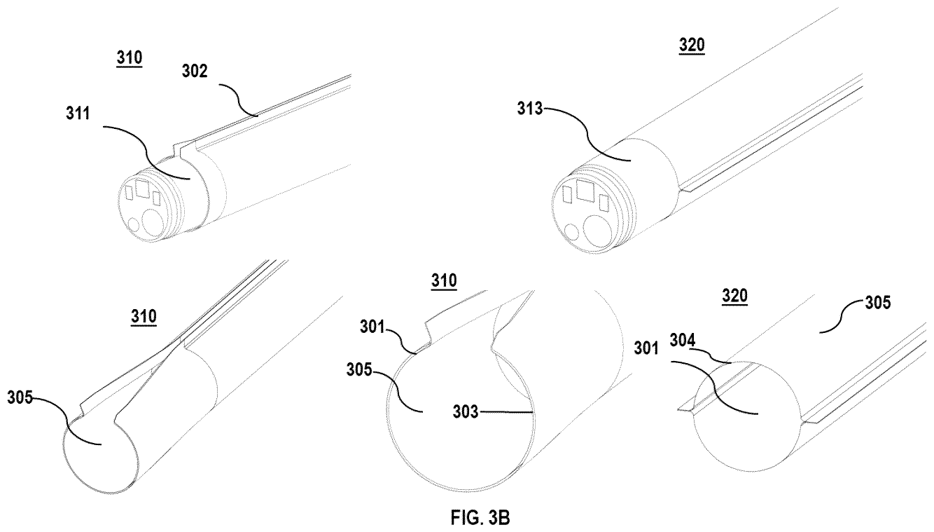

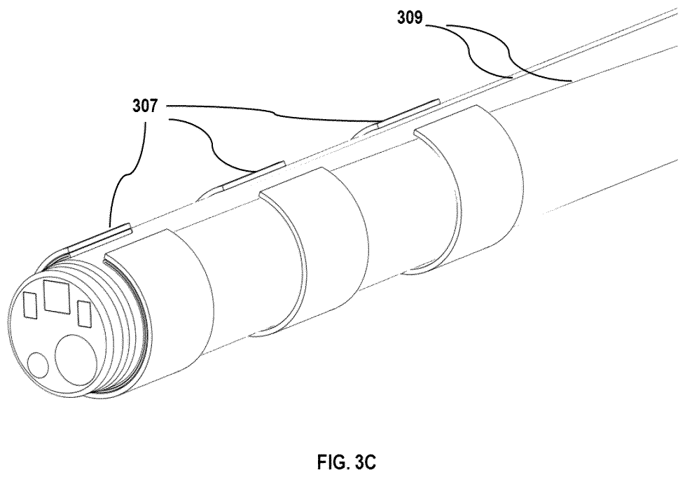

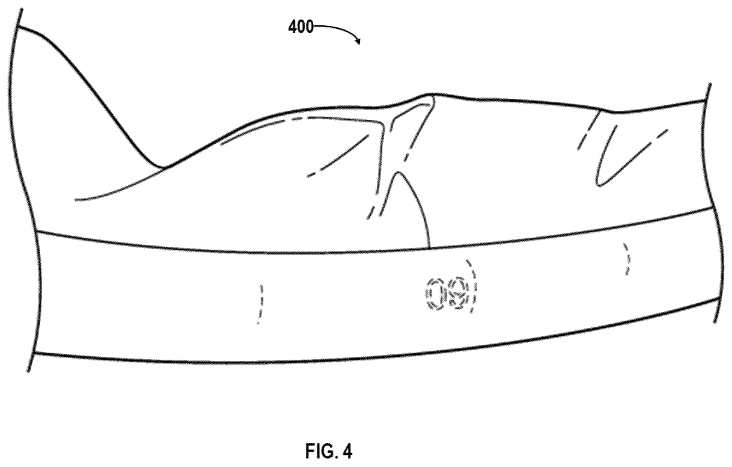

FIGS. 3A-3B shows examples 300 of another expandable overtube comprising fold features. The overtube 300 may comprise a temporary longitudinal seam 302. In some cases, the overtube may be constructed so as to roll or fold along its longitudinal axis to create a temporary lumen (or partial lumen with split) 305 for engaging a first scope (e.g., smaller scope 311 with a smaller diameter) for the initial intubation (e.g., standard colonoscope).

As illustrated in FIG. 3B, the overtube may fold to engage the standard colonoscope shaft 311 during the initial intubation 310. The layflat overtube construction may provide a geometry that can wrap around the colonoscope shaft for entry into the anatomy. By wrapping around the standard colonoscope during initial intubation, the outer diameter of the layflat overtube is reduced. In some cases, the fold or roll of the layflat overtube may form a lumen that can be positioned at the tip of the scope, or at defined positions along the shaft of the scope. The fold or roll features can be at discrete locations or may be continuous. FIG. 3C shows an example of one or more clips features 307 at discrete locations along the length that form the overtube into a substantially tubular lumen for initial intubation. The fold or roll features 307 may beneficially adjust the dimension of the tubular lumen such as by holding the lumen against a smaller diameter scope at initial intubation then allowing the lumen to expand to accept a larger diameter scope.

Referring back to FIG. 3B, in some embodiments, the overtube may comprise a primary lumen 301 and a temporary lumen 305. The dimension (e.g., diameter) of each lumen may be adjustable. For instance, a diameter of the second lumen (e.g., primary lumen 301) is adjusted by the folding/unfolding of the first lumen (e.g., temporary lumen 305). During the process 320 of intubating a larger scope 313, the second lumen (e.g., primary lumen 301) may be adapted to receive the larger scope. In some cases, a resting state of the layflat tube may be rolled along its longitudinal axis such that the roll forms a temporary lumen 305 through which a first scope (e.g., standard colonoscope 311) can be inserted through (shown in FIG. 3B). The smaller colonoscope 311 may be engaged with the outer surface 303 of the temporary lumen 305 of layflat, as opposed to its inner surfaces. The passage diameter of the primary lumen 301 is adjusted or determined at least by the temporary lumen 305. The larger scope may be inserted through the primary lumen 301 by contacting an inner surface 304 of the overtube or by unfolding the temporary lumen 305. The assembled overtube and standard colonoscope 311 may together result in a smaller introduction diameter (outer diameter) than if the colonoscope were placed inside the overtube.

The temporary lumen 305 may engage with a first scope (e.g., standard colonoscope or smaller scope) for initial intubation 310. Once the assembled first scope and overtube reaches a target site as described in FIG. 1, the first scope may be withdrawn and a second scope 313 (e.g., Gastroscope or larger scope) may be placed through the primary lumen of the overtube until it reaches the target site 320.

In some embodiments, a dimension of the tubular structure of the overtube device may be adjustable based on foldable construction of the tube. For instance, the primary lumen 301 may be expanded to accept a larger scope by unfolding the layflat primary lumen construction. In some cases, the edges 309 of the folded construction may be releasably coupled (to form the roll). In some cases, the edges of the folded construction may be separable but actively engaged. Alternatively, the edges of the folded construction may be separate but passively positioned relative to one another. The folds features may comprise passive engagement features, active engagement features or a combination of both and various other features.

In some cases, passive fold features (passive engagement features) may be employed to create the desired fold in a resting state. For example, passive fold features may include one or more split rings (e.g., split ring clip 205 in FIG. 2) positioned along the length of the layflat tube, whose resting state is closed. FIG. 3C shows an example of one or more clips features 307 that form the overtube into a substantially tubular lumen for initial intubation. In another example, the passive fold features may include a separable or peelable bi-lumen where each lumen is positively attached to one edge of the layflat and where separating the lumen from one another facilitates unfolding of the layflat primary lumen construction. In a further example, the passive fold features may include a separable or peelable thermal bond between the edges of the layflat tube (or features attached to the layflat tube). Various other passive features such as a slidably positioned ring or disc that can be dislodged from the layflat tube may also be utilized to allow expansion of the overtube.

In some cases, active engaged features or active engagement features of the overtube may be utilized to create a desired fold through energy. For example, the active engaged features may include one or more magnets positioned along the edges of the layflat tube that are attracted to one another, or a ferrous material on the opposing side. In another example, the active engaged features may include a cable or thread traversing one lumen before crossing over to be anchored in the adjacent lumen on the opposing layflat edge such that tension in the cable pulls the layflat edges towards one another to engage the scope. In a further example, the active engaged features may include a cable or thread that passes through eyelets alternating edges of the layflat tube, but is not anchored. Removal of the cable or thread releases the layflat tube to unfold. Other active engagement features such as positive or negative pressure applied to a closed volume along the layflat tube such that either positive or negative pressure, or a combination of both provides the stimulus to fold or unfold the layflat lumen may also be utilized. It should be noted that the above examples are for illustration purpose only and are not intended to be limiting.

The flexible overtube can be formed of any suitable materials such as polyurethane, polypropylene or polyethylene material. In some cases, materials (e.g., polyurethane) may be selected so it is easier to bond and readily creates seams via heat or radio-frequency welding. The various fold features (e.g., slit ring clip 205) can be formed any suitable materials such as a polyurethane, polypropylene, polyethylene, polycarbonate or any biocompatible semi rigid material that can handle the strain required during insertion of the larger colonoscope.

During intubation, the assembled colonoscope and overtube may be advanced together through the colon to the target site. When the target site is reached, the overtube balloon may be inflated. The balloon inflation may allow the overtube to anchor to the colon wall and the overtube can be pulled proximally to reduct (e.g., shorten and straighten) the colon. The first scope (e.g., colonoscope) may be removed from the colon and a second scope (e.g., Gastroscope) may be placed through the primary lumen of the overtube up to the target site.

In some cases, inflation of the balloon may act as a release mechanism for the temporary lumen. For instance, depending on the fold features of the overtube, inflation of the balloon may cause deformation of the split ring at the distal end of the overtube, initial separation of the peelable multi-lumen, initial separation of the peelable thermal bond, displacement of the magnets to break their attraction, displacement of the thread such that it is no longer engaged with the eyelets, allowing the primary lumen to expand, and the like. The initial engagement with the first scope (e.g., colonoscope) can be released via various other mechanisms that may or may not relate to the balloon inflation. For instance, fluid flow through the fill lumen may displace the magnets to eliminate the bond, or to position the magnets into a state of repulsion with one another to create a positive separation state.

In alternative embodiments, the overtube device may include a concentric, radially expanding and rigidizing overtube. The overtube device may comprise concentric tubular structures with a rigidizing medium in between. The concentric tubes may have a smaller diameter for primary intubation. After placing the tubes at the target site, the inner tube may be pressurized which results in radial expansion of the inner and outer tubes. While the inner tube is pressurized, the space between the inner and outer tube may be placed under vacuum such that the rigidizing medium between the inner and outer tubes locks into place with respect to the surfaces of the expanded inner tube and the outer tube. After rigidizing the structure, the inner tube may be depressurized and the rigid nature of the structure prevents radial collapse. The rigidizing medium can include any suitable materials or combinations of materials including, but not limited to, thin films with overlapping edges, braided structures made from metal or plastic filaments, foams having a porosity and surface finish that facilitates rigidization or any other granular mediums.

In alternative embodiments, the overtube device may vary the dimension of the tubular structure employing a shrinkable construction. In some cases, the overtube device may comprise a layflat overtube that is sealed on its distal end such that the internal volume can be placed under vacuum to reduce the external profile of the overtube. The reduced external profile is used to ease the insertion of the overtube into the anatomy and the vacuum is released prior to exchanging the first scope (e.g., standard colonoscope) for the larger second scope (e.g., gastroscope or robotic scope).

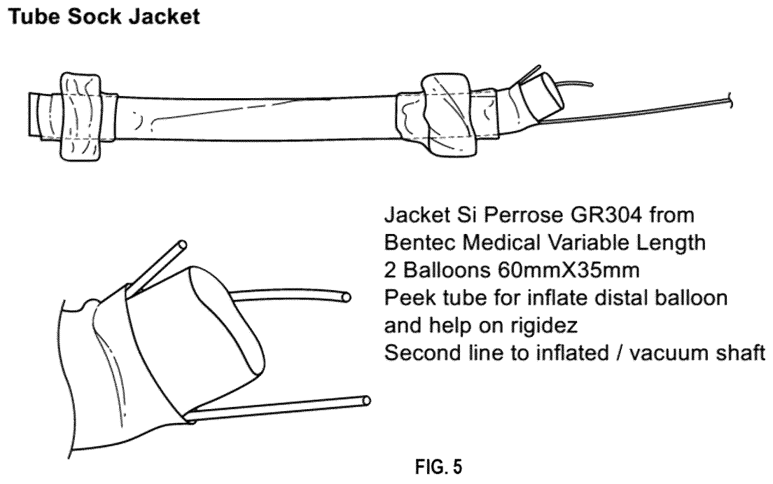

FIG. 4 and FIG. 5 show an example of an overtube device 400 including a shrinkable overtube. FIG. 4 shows an example of an overtube 400 providing for volume reduction via internal vacuum during initial intubation. The overtube may utilize vacuum to remove internal volume. As shown in the example, the inner space between lumen layers may be reduced by vacuum. FIG. 5 shows an example of an overtube device including a shrinkable overtube.

In alternative embodiments, the overtube device may comprise a multilumen construction. In some cases, the overtube device may comprise thin-wall multilumen construction with suture folding.

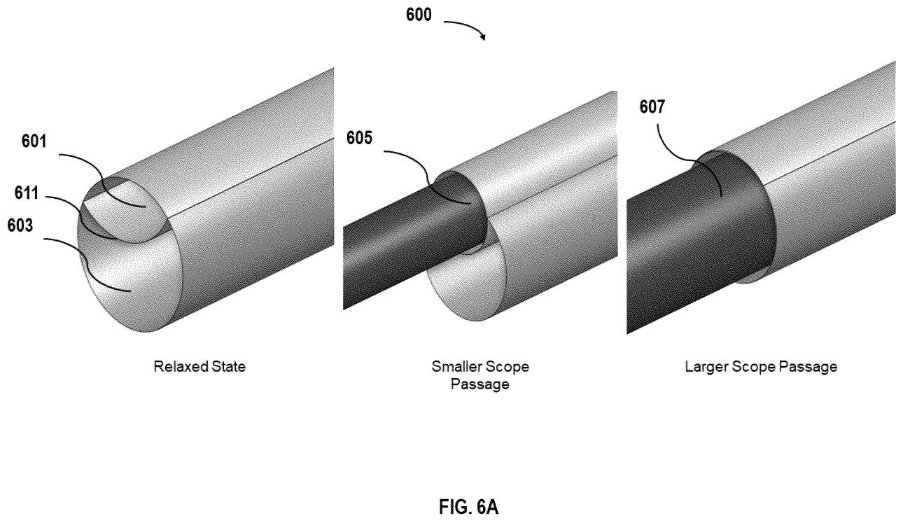

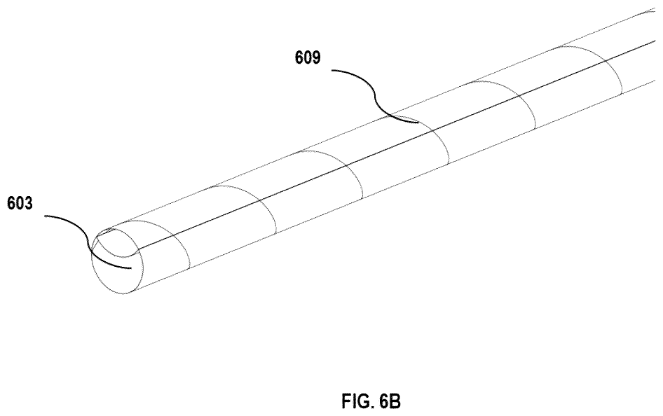

FIG. 6A shows an example of an overtube device 600 with multi-lumen construction. In the illustrated example, the overtube may comprise a layflat tube construction with a dividing layer 611 that separates a smaller channel 601 from a larger channel 603. The smaller channel may form a first lumen accepting a smaller scope, the larger channel may form a second lumen accepting a larger scope, where the first lumen and the second lumen co-exit but the dimension is variable. As shown in the example, the smaller channel 601 may be used to pass through a first scope with a smaller size (e.g., colonoscope) 605 and the larger channel 603 may be capable of passing a second scope with a larger size 607. The flexibility of the lumen separator (e.g., dividing layer 611) allows the smaller channel to open for the smaller colonoscope for initial intubation and then collapse when the smaller colonoscope is not engaged. Additionally, a suture thread may be positioned and tensioned to retain the larger lumen in a collapsed state until the physician desires to expand larger lumen for passing the larger scope. The example in FIG. 6B illustrates an example suture thread 609 following a helical pattern to retain the larger lumen. Various other patterns of suture positioning relative to the lumen can be utilized.

### Flexible Endoscope

The intubation method and devices can be utilized in a robotic endoscopic system. In some cases, the intubation method and devices herein may be applied to robotic endoscope that is single-use or reusable. Endoscopes are traditionally made to be re-usable, which may require thorough cleaning, dis-infection, and/or sterilization after each procedure. In most cases, cleaning, dis-infection, and sterilization may be aggressive processes to kill germs and/or bacteria. Such procedures may also be harsh on the endoscopes themselves. Therefore, the designs of such re-usable endoscopes can often be complicated, especially to ensure that the endoscopes can survive such harsh cleaning, dis-infection, and sterilization protocols. Periodical maintenance and repairs for such re-usable endoscopes may often be needed.

Low cost, disposable medical devices designated for a single-use have become popular for instruments that are difficult to clean properly. Single-use, disposable devices may be packaged in sterile wrappers to avoid the risk of pathogenic cross-contamination of diseases such as HIV, hepatitis, and other pathogens. Hospitals generally welcome the convenience of single-use disposable products because they no longer have to be concerned with product age, overuse, breakage, malfunction, and sterilization. Traditional endoscopes often include a handle that operators use to maneuver the endoscope. For single-use endoscopes, the handle usually encloses the camera, expensive electronics, and mechanical structures at proximal end in order to transmit the video and allow the users to maneuver the endoscope via a user interface. This may lead to high cost of the handle for a single-use endoscope.

In some embodiments, the overtube device and method as provided herein may be utilized to intubate a flexible endoscope that may be single-use or disposable. Alternatively, flexible endoscope may be reusable. FIG. 7 illustrates an example of a flexible endoscope 1000, in accordance with some embodiments of the present disclosure. As shown in FIG. 7, the flexible endoscope 1000 may comprise a handle/proximal portion 1009 and a flexible elongate member to be inserted inside of a subject. The flexible elongate member can be the same as the one described above. In some embodiments, the flexible elongate member may comprise a proximal shaft (e.g., insertion shaft 1001), steerable tip (e.g., tip 1005), a steerable section (active bending section 1003) and an anti-prolapse passive section 1004. The active bending section, an anti-prolapse passive section and the proximal shaft section can be the same as those described elsewhere herein. The endoscope 100 may also be referred to as steerable catheter assembly as described elsewhere herein. In some cases, the endoscope 100 may be a single-use robotic endoscope. In some cases, the entire catheter assembly may be disposable. In some cases, at least a portion of the catheter assembly may be disposable. In some cases, the entire endoscope may be released from an instrument driving mechanism and can be disposed of. In some embodiment, the endoscope may contain varying levels of stiffness along the shaft, as to improve functional operation.

The endoscope or steerable catheter assembly 1000 may comprise a handle portion 1009 that may include one or more components configured to process image data, provide power, or establish communication with other external devices. For instance, the handle portion may include a circuitry and communication elements that enables electrical communication between the steerable catheter assembly 1000 and an instrument driving mechanism (not shown), and any other external system or devices. In another example, the handle portion 1009 may comprise circuitry elements such as power sources for powering the electronics (e.g., camera, electromagnetic sensor and LED lights) of the endoscope.

The one or more components located at the handle may be optimized such that expensive and complicated components may be allocated to the robotic support system, a hand-held controller or an instrument driving mechanism thereby reducing the cost and simplifying the design the disposable endoscope. The handle portion or proximal portion may provide an electrical and mechanical interface to allow for electrical communication and mechanical communication with the instrument driving mechanism. The instrument driving mechanism may comprise a set of motors that are actuated to rotationally drive a set of pull wires of the catheter. The handle portion of the catheter assembly may be mounted onto the instrument drive mechanism so that its pulley/capstans assemblies are driven by the set of motors. The number of pulleys may vary based on the pull wire configurations. In some cases, one, two, three, four, or more pull wires may be utilized for articulating the flexible endoscope or catheter.

The handle portion may be designed allowing the robotic endoscope to be disposable at reduced cost. For instance, classic manual and robotic endoscopes may have a cable in the proximal end of the endoscope handle. The cable often includes illumination fibers, camera video cable, and other sensors fibers or cables such as electromagnetic (EM) sensors, or shape sensing fibers. Such complex cable can be expensive adding to the cost of the endoscope. The provided robotic endoscope may have an optimized design such that simplified structures and components can be employed while preserving the mechanical and electrical functionalities. In some cases, the handle portion of the robotic endoscope may employ a cable-free design while providing a mechanical/electrical interface to the catheter.

The electrical interface (e.g., printed circuit board) may allow image/video data and/or sensor data to be received by the communication module of the instrument driving mechanism and may be transmitted to other external devices/systems. In some cases, the electrical interface may establish electrical communication without cables or wires. For example, the interface may comprise pins soldered onto an electronics board such as a printed circuit board (PCB). For instance, receptacle connector (e.g., the female connector) is provided on the instrument driving mechanism as the mating interface. This may beneficially allow the endoscope to be quickly plugged into the instrument driving mechanism or robotic support without utilizing extra cables. Such type of electrical interface may also serve as a mechanical interface such that when the handle portion is plugged into the instrument driving mechanism, both mechanical and electrical coupling is established. Alternatively or in addition to, the instrument driving mechanism may provide a mechanical interface only. The handle portion may be in electrical communication with a modular wireless communication device or any other user device (e.g., portable/hand-held device or controller) for transmitting sensor data and/or receiving control signals.

In some cases, the handle portion 1009 may comprise one or more mechanical control modules such as lure 1011 for interfacing the irrigation system/aspiration system. In some cases, the handle portion may include lever/knob for articulation control. Alternatively, the articulation control may be located at a separate controller attached to the handle portion via the instrument driving mechanism.

The endoscope may be attached to a robotic support system or a hand-held controller via the instrument driving mechanism. The instrument driving mechanism may be provided by any suitable controller device (e.g., hand-held controller) that may or may not include a robotic system. The instrument driving mechanism may provide mechanical and electrical interface to the steerable catheter assembly 1000. The mechanical interface may allow the steerable catheter assembly 1000 to be releasably coupled to the instrument driving mechanism. For instance, the handle portion of the steerable catheter assembly can be attached to the instrument driving mechanism via quick install/release means, such as magnets, spring-loaded levels and the like. In some cases, the steerable catheter assembly may be coupled to or released from the instrument driving mechanism manually without using a tool. Details about the instrument driving mechanism are described later herein.

In the illustrated example, the distal tip of the catheter or endoscope shaft is configured to be articulated/bent in two or more degrees of freedom to provide a desired camera view or control the direction of the endoscope. As illustrated in the example, imaging device (e.g., camera), position sensors (e.g., electromagnetic sensor) 1007 is located at the tip of the catheter or endoscope shaft 1005. For example, line of sight of the camera may be controlled by controlling the articulation of the active bending section 1003. In some instances, the angle of the camera may be adjustable such that the line of sight can be adjusted without or in addition to articulating the distal tip of the catheter or endoscope shaft. For example, the camera may be oriented at an angle (e.g., tilt) with respect to the axial direction of the tip of the endoscope with aid of an optimal component.

The distal tip 1005 may be a rigid component that allow for positioning sensors such as electromagnetic (EM) sensors, imaging devices (e.g., camera) and other electronic components (e.g., LED light source) being embedded at the distal tip.

In real-time EM tracking, the EM sensor comprising of one or more sensor coils embedded in one or more locations and orientations in the medical instrument (e.g., tip of the endoscopic tool) measures the variation in the EM field created by one or more static EM field generators positioned at a location close to a patient. The location information detected by the EM sensors is stored as EM data. The EM field generator (or transmitter), may be placed close to the patient to create a low intensity magnetic field that the embedded sensor may detect. The magnetic field induces small currents in the sensor coils of the EM sensor, which may be analyzed to determine the distance and angle between the EM sensor and the EM field generator. For example, the EM field generator may be positioned close to the patient torso during procedure to locate the EM sensor position in 3D space or may locate the EM sensor position and orientation in 5D or 6D space. This may provide a visual guide to an operator when driving the endoscope towards the target site.

The endoscope may have a unique design in the elongate member. In some cases, the active bending section 1003, the anti-prolapse passive section and the proximal shaft of the endoscope may consist of a single tube that incorporates a series of cuts (e.g., reliefs, slits, etc.) along its length to allow for improved flexibility, a desirable stiffness as well as the anti-prolapse feature (e.g., features to define a minimum bend radius).

As described above, the active bending section 1003 may be designed to allow for bending in two or more degrees of freedom (e.g., articulation). A greater bending degree such as 180 and 270 degrees (or other articulation parameters for clinical indications) can be achieved by the unique structure of the active bending section while kinking or prolapse may be prevented by the passive section following the active bending section. In some cases, the active bending section and/or the passive section may be fabricated separately as a modular component and assembled to the proximal shaft. In some cases, the cut patterns of the active bending and passive sections may be different such that at least the minimum bend radius of the two sections may be different. In some cases, a variable minimum bend radius along the axial axis of the elongate member may be provided such that an active bending section or the passive section may comprise two or more different minimum bend radii.

The articulation of the endoscope may be controlled by applying force to the distal end of the endoscope via one or multiple pull wires. The one or more pull wires may be attached to the distal end of the endoscope. In the case of multiple pull wires, pulling one wire at a time may change the orientation of the distal tip to pitch up, down, left, right or any direction needed. In some cases, the pull wires may be anchored at the distal tip of the endoscope, running through the bending section, and entering the handle where they are coupled to a driving component (e.g., pulley). This handle pulley may interact with an output shaft from the robotic system.

In some embodiments, the proximal end or portion of one or more pull wires may be operatively coupled to various mechanisms (e.g., gears, pulleys, capstans, etc.) in the handle portion of the catheter assembly. The pull wire may be a metallic wire, cable or thread, or it may be a polymeric wire, cable or thread. The pull wire can also be made of natural or organic materials or fibers. The pull wire can be any type of suitable wire, cable or thread capable of supporting various kinds of loads without deformation, significant deformation, or breakage. The distal end/portion of one or more pull wires may be anchored or integrated to the distal portion of the catheter, such that operation of the pull wires by the control unit may apply force or tension to the distal portion which may steer or articulate (e.g., up, down, pitch, yaw, or any direction in-between) at least the distal portion (e.g., flexible section) of the catheter.

The pull wires may be made of any suitable material such as stainless steel (e.g., SS316), metals, alloys, polymers, nylons or biocompatible material. Pull wires may be a wire, cable or a thread. In some embodiments, different pull wires may be made of different materials for varying the load bearing capabilities of the pull wires. In some embodiments, different sections of the pull wires may be made of different material to vary the stiffness and/or load bearing along the pull. In some embodiments, pull wires may be utilized for the transfer of electrical signals.

The proximal design may improve the reliability of the device without introducing extra cost allowing for a low-cost single-use endoscope. In another aspect of the invention, a single-use robotic endoscope is provided. The robotic endoscope may be a gastroscope and can be the same as the steerable catheter assembly as described elsewhere herein. Traditional endoscopes can be complex in design and are usually designed to be re-used after procedures, which require thorough cleaning, dis-infection, or sterilization after each procedure. The existing endoscopes are often designed with complex structures to ensure the endoscopes can endure the cleaning, dis-infection, and sterilization processes. The provided robotic endoscope can be a single-use endoscope that may beneficially reduce cross-contamination between patients and infections. In some cases, the robotic gastroscope may be delivered to the medical practitioner in a pre-sterilized package and are intended to be disposed of after a single-use.

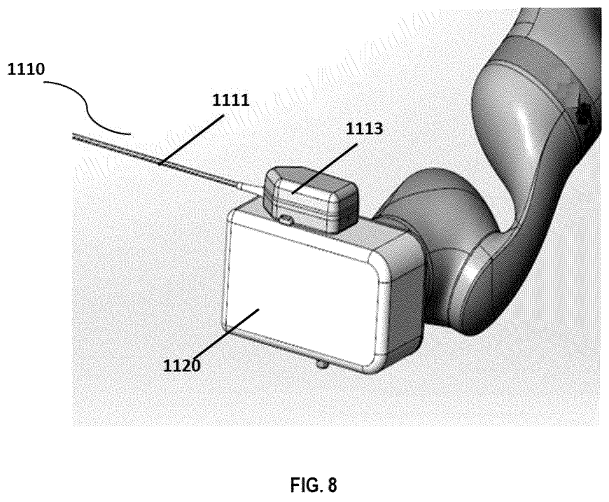

As shown in FIG. 8, a robotic gastroscope 1120 may comprise a handle portion 1113 and a flexible elongate member 1111. In some embodiments, the flexible elongate member 1111 may comprise a shaft, steerable tip, a steerable/active bending section and an anti-prolapse passive section. The robotic gastroscope 1120 can be the same as the steerable catheter assembly as described in FIG. 7. The robotic gastroscope may be a single-use robotic endoscope. In some cases, only the catheter may be disposable. In some cases, at least a portion of the catheter may be disposable. In some cases, the entire robotic gastroscope may be released from the instrument driving mechanism and can be disposed of. In some cases, the gastroscope may contain varying levels of stiffness along its shaft, as to improve functional operation. In some cases, a minimum bend radius along the shaft may vary so that the kink resistance or anti-prolapse capability may be configurable along the length.

The robotic gastroscope can be releasably coupled to an instrument driving mechanism 1120. The instrument driving mechanism 1120 may be mounted to the arm of the robotic support system or to any actuated support system as described elsewhere herein. The instrument driving mechanism may provide mechanical and electrical interface to the robotic gastroscope 1110. The mechanical interface may allow the robotic gastroscope 1110 to be releasably coupled to the instrument driving mechanism. For instance, the handle portion of the robotic gastroscope can be attached to the instrument driving mechanism via quick install/release means, such as magnets and spring-loaded levels. In some cases, the robotic gastroscope may be coupled or released from the instrument driving mechanism manually without using a tool.

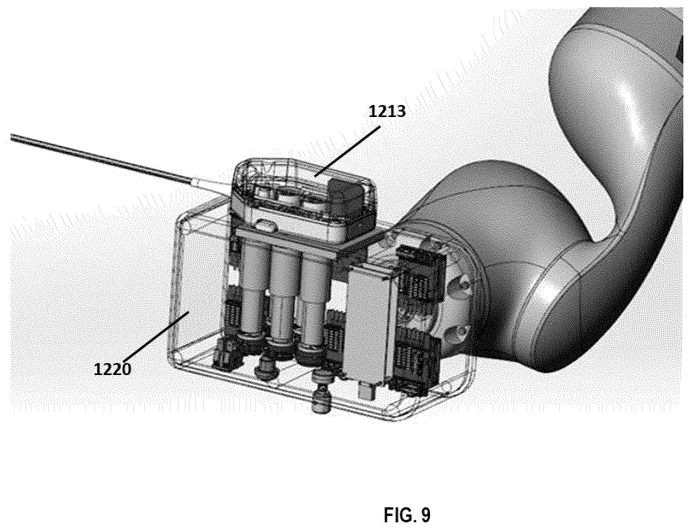

FIG. 9 shows an example of an instrument driving mechanism 1220 providing mechanical interface to the handle portion 1213 of the robotic endoscope. As shown in the example, the instrument driving mechanism 1220 may comprise a set of motors that are actuated to rotationally drive a set of pull wires of the flexible endoscope or catheter. The handle portion 1213 of the catheter assembly may be mounted onto the instrument drive mechanism so that its pulley assemblies or capstans are driven by the set of motors. The number of pulleys may vary based on the pull wire configurations. In some cases, one, two, three, four, or more pull wires may be utilized for articulating the flexible endoscope or catheter.

The handle portion may be designed allowing the robotic gastroscope to be disposable at reduced cost. For instance, classic manual and robotic gastroscopes may have a cable in the proximal end of the gastroscope handle. The cable often includes illumination fibers, camera video cable, and other sensors fibers or cables such as electromagnetic (EM) sensors, or shape sensing fibers. Such complex cable can be expensive, adding to the cost of the gastroscope. The provided robotic gastroscope may have an optimized design such that simplified structures and components can be employed while preserving the mechanical and electrical functionalities. In some cases, the handle portion of the robotic gastroscope may employ a cable-free design while providing a mechanical/electrical interface to the catheter.

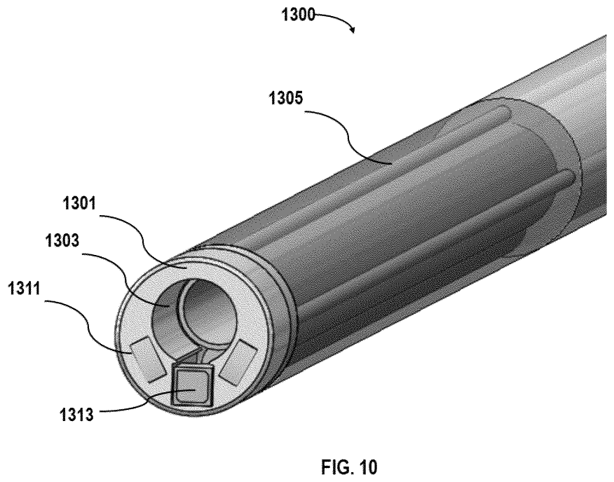

FIG. 10 shows an example of a distal tip 1300 of an endoscope. In some cases, the distal portion or tip of the catheter 1300 may be substantially flexible such that it can be steered into one or more directions (e.g., pitch, yaw). The catheter may comprise a tip portion, bending section, and insertion shaft. In some embodiments, the catheter may have variable bending stiffness along the longitudinal axis direction. For instance, the catheter may comprise multiple sections having different bending stiffness (e.g., flexible, semi-rigid, and rigid). The bending stiffness may be varied by selecting materials with different stiffness/rigidity, varying structures in different segments (e.g., cuts, patterns), adding additional supporting components or any combination of the above. In some embodiments, the catheter may have variable minimum bend radius along the longitudinal axis direction. The selection of different minimum bend radius at different location long the catheter may beneficially provide anti-prolapse capability while still allow the catheter to reach hard-to-reach regions. In some cases, a proximal end of the catheter needs not be bent to a high degree thus the proximal portion of the catheter may be reinforced with additional mechanical structure (e.g., additional layers of materials) to achieve a greater bending stiffness. Such design may provide support and stability to the catheter. In some cases, the variable bending stiffness may be achieved by using different materials during extrusion of the catheter. This may advantageously allow for different stiffness levels along the shaft of the catheter in an extrusion manufacturing process without additional fastening or assembling of different materials.

The distal portion of the catheter may be steered by one or more pull wires 1305. The distal portion of the catheter may be made of any suitable material such as co-polymers, polymers, metals or alloys such that it can be bent by the pull wires. In some embodiments, the proximal end or terminal end of one or more pull wires 1305 may be coupled to a driving mechanism (e.g., gears, pulleys, capstan etc.) via the anchoring mechanism as described above.

The pull wire 1305 may be a metallic wire, cable or thread, or it may be a polymeric wire, cable or thread. The pull wire 1305 can also be made of natural or organic materials or fibers. The pull wire 1305 can be any type of suitable wire, cable or thread capable of supporting various kinds of loads without deformation, significant deformation, or breakage. The distal end or portion of one or more pull wires 1305 may be anchored or integrated to the distal portion of the catheter, such that operation of the pull wires by the control unit may apply force or tension to the distal portion which may steer or articulate (e.g., up, down, pitch, yaw, or any direction in-between) at least the distal portion (e.g., flexible section) of the catheter.

The catheter may have a dimension so that one or more electronic components can be integrated to the catheter. For example, the outer diameter of the distal tip may be around 4 to 4.4 millimeters (mm), and the diameter of the working channel 1303 may be around 2 mm such that one or more electronic components can be embedded into the wall of the catheter. However, it should be noted that based on different applications, the outer diameter can be in any range smaller than 4 mm or greater than 4.4 mm, and the diameter of the working channel can be in any range according to the tool dimension or specific application.

The one or more electronic components may comprise an imaging device, illumination device or sensors. In some embodiments, the imaging device may be a video camera 1313. The imaging device may comprise optical elements and image sensor for capturing image data. The image sensors may be configured to generate image data in response to wavelengths of light. A variety of image sensors may be employed for capturing image data such as complementary metal oxide semiconductor (CMOS) or charge-coupled device (CCD). The imaging device may be a low-cost camera. In some cases, the image sensor may be provided on a circuit board. The circuit board may be an imaging printed circuit board (PCB). The PCB may comprise a plurality of electronic elements for processing the image signal. For instance, the circuit for a CCD sensor may comprise A/D converters and amplifiers to amplify and convert the analog signal provided by the CCD sensor. Optionally, the image sensor may be integrated with amplifiers and converters to convert analog signal to digital signal such that a circuit board may not be required. In some cases, the output of the image sensor or the circuit board may be image data (digital signals) can be further processed by a camera circuit or processors of the camera. In some cases, the image sensor may comprise an array of optical sensors.

The illumination device may comprise one or more light sources 1311 positioned at the distal tip. The light source may be a light-emitting diode (LED), an organic LED (OLED), a quantum dot, or any other suitable light source. In some cases, the light source may be miniaturized LED for a compact design or Dual Tone Flash LED Lighting.

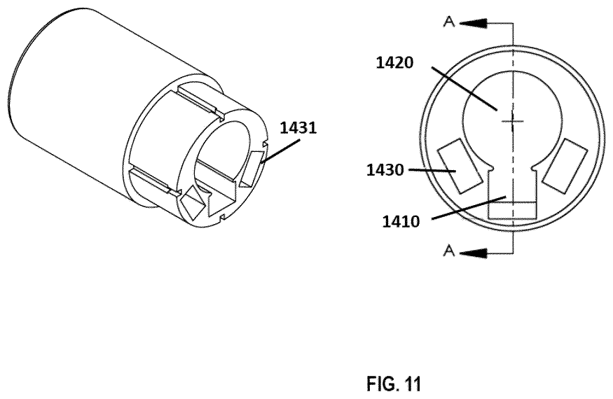

The imaging device and the illumination device may be integrated to the catheter. For example, the distal portion of the catheter may comprise suitable structures matching at least a dimension of the imaging device and the illumination device. The imaging device and the illumination device may be embedded into the catheter. FIG. 11 shows an example distal portion of the catheter with integrated imaging device and the illumination device. A camera may be located at the distal portion. The distal tip may have a structure to receive the camera, illumination device and/or the location sensor. For example, the camera may be embedded into a cavity 1410 at the distal tip of the catheter. The cavity 1410 may be integrally formed with the distal portion of the cavity and may have a dimension matching a length/width of the camera such that the camera may not move relative to the catheter. The camera may be adjacent to the working channel 1420 of the catheter to provide near field view of the tissue or the organs. In some cases, the attitude or orientation of the imaging device may be controlled by controlling a rotational movement (e.g., roll) of the catheter.

The power to the camera may be provided by a wired cable. In some cases, the cable wire may be in a wire bundle providing power to the camera as well as illumination elements or other circuitry at the distal tip of the catheter. The camera and/or light source may be supplied with power from a power source located at the handle portion via wires, copper wires, or via any other suitable means running through the length of the catheter. In some cases, real-time images or video of the tissue or organ may be transmitted to an external user interface or display wirelessly. The wireless communication may be WiFi, Bluetooth, RF communication or other forms of communication. In some cases, images or videos captured by the camera may be broadcasted to a plurality of devices or systems. In some cases, image and/or video data from the camera may be transmitted down the length of the catheter to the processors situated in the handle portion via wires, copper wires, or via any other suitable means. The image or video data may be transmitted via the wireless communication component in the handle portion to an external device/system. In some cases, the system may be designed such that no wires are visible or exposed to operators.

In conventional endoscopy, illumination light may be provided by fiber cables that transfer the light of a light source located at the proximal end of the endoscope, to the distal end of the robotic endoscope. In some embodiments of the disclosure, miniaturized LED lights may be employed and embedded into the distal portion of the catheter to reduce the design complexity. In some cases, the distal portion may comprise a structure 1430 having a dimension matching a dimension of the miniaturized LED light source. As shown in the illustrated example, two cavities 1430 may be integrally formed with the catheter to receive two LED light sources. For instance, the outer diameter of the distal tip may be around 4 to 4.4 millimeters (mm) and diameter of the working channel of the catheter may be around 2 mm such that two LED light sources may be embedded at the distal end. The outer diameter can be in any range smaller than 4 mm or greater than 4.4 mm, and the diameter of the working channel can be in any range according to the tool's dimensional or specific application. Any number of light sources may be included. The internal structure of the distal portion may be designed to fit any number of light sources.

In some cases, each of the LEDs may be connected to power wires which may run to the proximal handle. In some embodiment, the LEDs may be soldered to separated power wires that later bundle together to form a single strand. In some embodiments, the LEDs may be soldered to pull wires that supply power. In other embodiments, the LEDs may be crimped or connected directly to a single pair of power wires. In some cases, a protection layer such as a thin layer of biocompatible glue may be applied to the front surface of the LEDs to provide protection while allowing light emitted out. In some cases, an additional cover 1431 may be placed at the forwarding end face of the distal tip providing precise positioning of the LEDs as well as sufficient room for the glue. The cover 1431 may be composed of transparent material matching the refractive index of the glue so that the illumination light may not be obstructed.

The working channel (e.g., working channel 1303, 1420) may be designed to provide protection for the internal components such as flexible instruments (e.g., needle, forceps, etc.). When flexible instruments pass through a conventional working channel, they may be abstracted by the working channel due to kinking, ovalizing and/or high friction force. The working channel herein may advantageously address the above drawbacks by providing a high hoop strength and a capability of achieving low bend radius. The working channel may also be designed to provide low friction in the inner surface.

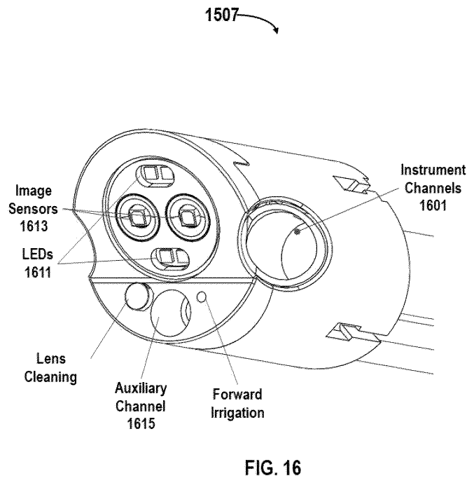

FIG. 16 shows another example of a tip 1507 for a robotic endoscope device. The tip 1507 may comprise image sensors 1613, light sources 1611 same as those as described above. The tip may also further other features such as lens cleaning, forward irrigation to provide a clear view of the camera. The working channel (e.g., instrument channel 1601, auxiliary channel 1615) may be designed to provide protection for the internal components such as flexible instruments (e.g., suturing instrument, forceps, etc.). When flexible instruments pass through a conventional working channel, they may be obstructed by the working channel due to kinking, ovalizing and/or high friction force. The working channel may provide a high hoop strength and a capability of achieving low bend radius. The working channel may also be designed to provide low friction in the inner surface. The suturing instrument as described herein may be passed through the working channel and advanced over the distal tip of the endoscope or retracted back into the working channel.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A device for intubating an endoscope into a subject, the device comprising:
a flexible overtube comprising features to form a first lumen for passing through a first scope during intubation, wherein the first lumen is deformable to reduce a volume of the first lumen for creating a second lumen for passing through a second scope, wherein the second scope has a diameter greater than a diameter of the first scope and wherein a diameter of the first lumen is smaller than a diameter of the second lumen.

2. The device of claim 1, wherein the features comprise an expandible tubular structure to adjust a dimension of the first lumen or the second lumen.

3. The device of claim 2, wherein the expandible tubular structure is a layflat tube construction with a pleat along an axial direction.

4. The device of claim 2, wherein the expandible tubular structure is a foldable layflat tube construction.

5. The device of claim 4, wherein the foldable layflat tube construction has separable edges that are engaged with aid of one or more active engagement features.

6. The device of claim 4, wherein the foldable layflat tube construction reduces a diameter of the first lumen to create the second lumen with aid of one or more passive engagement features.

7. The device of claim 1, wherein the first lumen and the second lumen are two channels separated by a lumen separator of the flexible overtube.

8. The device of claim 1, wherein the second scope is a robotic scope.

9. The device of claim 8, wherein the robotic scope comprises a handle portion releasably coupled to a robotic support and wherein the handle portion of the robotic scope is coupled to the robotic support after the robotic scope is inserted through the second lumen assuming a tortuous shape.

10. The device of claim 9, wherein the robotic scope is initialized by removing slack in one or more pull wires for controlling articulation of a bending section of the robotic scope while the bending section conforms to an internal environment within the subject.

11. A method for intubating a robotic endoscope into a subject, the method comprising:
(a) performing an initial intubation to reach a target site within a body of the subject with a first scope and an overtube device, wherein the first scope is engaged with a first lumen of the overtube device;
(b) withdrawing the first scope and inserting a second scope into a second lumen of the overtube device to reach the target site, wherein the second scope is a robotic scope having a diameter greater than a diameter of the first scope; and
(c) coupling a handle portion of the second scope to an instrument driving mechanism (IDM) and performing initialization of the second scope while the second scope is within the body of the subject.

12. The method of claim 11, wherein the initialization comprises removing a slack in one or more pull wires of the second scope.

13. The method of claim 12, wherein the one or more pull wires are driven by the IDM to control an articulation of a bending section of the second scope in one or more degrees of freedom.

14. The method of claim 13, further comprising monitoring a tension in one or more pull wires corresponding to one degree of freedom.

15. The method of claim 14, further comprising comparing a difference of the tension in the one or more pull wires against a predetermined threshold.

16. The method of claim 15, further comprising controlling one or more actuators of the IDM based on the tension or the difference of the tension.

17. The method of claim 11, wherein the first lumen is deformable for creating a second lumen.

18. The method of claim 11, wherein the overtube device comprises an expandible tubular structure to adjust a dimension of the first lumen or the second lumen.

19. The method of claim 18, wherein the expandible tubular structure is a layflat tube construction with a pleat along an axial direction.

20. The method of claim 11, wherein the first lumen and the second lumen are two channels separated by a lumen separator of the overtube device.
